(19) United States
(12) Patent Application Publication
(10) Pub. No.: US 2025/0237633 A1
(43) Pub. Date: Jul. 24, 2025

(54) Multi-Sensor for Stationary Energy Storage Systems

(71) Applicant: Maarten P.J. Van Laere, Leuven (BE)

(72) Inventor: Maarten P.J. Van Laere, Leuven (BE)

(21) Appl. No.: 18/945,417

(22) Filed: Nov. 12, 2024

Related U.S. Application Data
(60) Provisional application No. 63/548,372, filed on Nov. 13, 2023.

(51) Int. Cl. G01N 33/00; H01M 10/48

(52) U.S. Cl. CPC G01N 33/0031; G01N 33/0006; G01N 33/0047; G01N 33/005; H01M 10/48

(57) ABSTRACT

The present invention is a multi-sensor that combines at least VOC and H2 detection into a single device. The sensor may also include additional features, such as: A technology that avoids drifting or a self-calibration mechanism that eliminates the need for in-field calibration; Optional dry contact and/or relay outputs to control external systems; Wired or wireless communication capabilities to connect to other systems, such as fire panels. The sensor can be used in a variety of stationary energy storage systems, including but not limited to: UPS systems; Energy storage systems; and Battery packs.

### TECHNICAL FIELD OF THE PRESENT INVENTION

The present invention relates generally to stationary energy storage systems. More specifically the present invention relates to a multi-sensor for stationary energy storage systems.

### BACKGROUND OF THE PRESENT INVENTION

The field of stationary energy storage systems has grown rapidly in recent years, driven by the increasing demand for renewable energy sources. These systems typically rely on batteries to store energy, and batteries can be a fire hazard if they are not properly managed.

One of the main challenges of managing battery safety is detecting off-gas events. Off-gas events occur when batteries release gases that can be flammable or toxic. There are three main types of off-gas events:
Overcharging: This occurs when batteries are charged beyond their capacity.
Battery leak: This occurs when batteries are damaged and allow electrolyte to leak out.
Hydrogen release: This occurs typically when there is a leak in liquid cooled batteries or when using lead acid batteries.

Traditionally, customers have relied on volatile organic compound (VOC) sensors to detect off-gas events from batteries. However, VOC sensors are only able to detect two of the three types of off-gas events: overcharging and battery leak. They cannot detect hydrogen release.

To detect hydrogen release, customers must install separate hydrogen (H2) sensors. However, these sensors have several drawbacks:
They are expensive to purchase and install.
They require annual in-field calibration.
They have a limited lifespan of 5 years or less.

While VOC sensors may have cross-sensitivity to Hydrogen they are unable to discriminate between hydrogen and VOCs. This is a major drawback when it comes to safety.

### SUMMARY OF THE INVENTION

The present invention is a multi-sensor that combines at least VOC and H2 detection into a single device. The sensor may also include additional features, such as:
A technology that avoids drifting or a self-calibration mechanism that eliminates the need for in-field calibration.
Optional dry contact and/or relay outputs to control external systems.
Wired or wireless communication capabilities to connect to other systems, such as fire panels.

The sensor can be used in a variety of stationary energy storage systems, including but not limited to:
UPS systems
Energy storage systems
Battery packs

### BRIEF DESCRIPTION OF THE DRAWINGS

The accompanying drawings, which are incorporated herein and form a part of the specification, illustrate the present invention and, together with the description, further serve to explain the principles of the present invention and to enable a person skilled in the pertinent art to make and use the present invention.

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 illustrates a possible embodiment of the sensor with its logical flow for processing H2 and VOC sensor data with possible other sensing data.

### DETAILED DESCRIPTION OF THE PRESENT INVENTION

In the following detailed description of the present invention of exemplary embodiments of the present invention, reference is made to the accompanying drawings (where like numbers represent like elements), which form a part hereof, and in which is shown by way of illustration specific exemplary embodiments in which the present invention may be practiced. These embodiments are described in sufficient detail to enable those skilled in the art to practice the present invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the present invention. However, it is understood that the present invention may be practiced without these specific details. In other instances, well-known structures and techniques known to one of ordinary skill in the art have not been shown in detail in order not to obscure the present invention. Referring to the figures, it is possible to see the various major elements constituting the apparatus of the present invention.

The multi-sensor of the present invention comprises at least the following:
A VOC sensor component that detects volatile organic compounds.
An H2 sensor component that detects hydrogen.
A technology or a self-calibration mechanism that calibrates the sensor without the need for human intervention or that avoids drifting like regular sensors face.
Optional dry contact or relay outputs that can be used to control external systems.
Wired or wireless communication capabilities that allow the sensor to connect to other systems, such as fire panels.

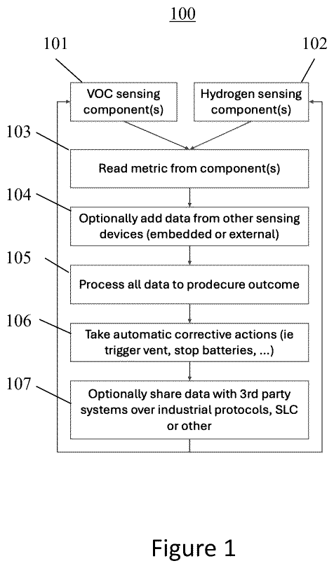

FIG. 1 illustrates a possible embodiment of the sensor with its logical flow for processing H2 and VOC sensor data with possible other sensing data 100.

The VOC sensor 101 can be any type of VOC sensor, such as but not limited to a semiconductor sensor or an electrochemical sensor.

The H2 sensor 102 can use any type of hydrogen detection method, such as a metal oxide semiconductor (MOS) sensor, an electrochemical sensor, spectrometer or any other type of detection technology.

The method for processing the information from VOC sensing components 101 and Hydrogen (H2) sensing components 102 starts with reading the metric data from the components 103. Optionally, the processing method can include a step of adding data from other embedded or external sensing devices 104.

A procedure is applied to process all data to generate an outcome 105. Automatic corrective actions are taken 106 such as triggering a vent or stopping batteries.

Optionally, data can be shared with 3rd party systems over industrial protocols, SLC or other communications systems.

The self-calibration mechanism can be any type of self-calibration mechanism, such as a mechanism that uses a known gas concentration to calibrate the sensor or a mechanism that uses the sensor's own output to calibrate itself. It can also use data from other sensors to compensate for drifting. Or use a different technology to avoid drifting all-together.

The dry contact or relay outputs can be used to control external systems, such as vents or alarms. The communication capabilities can be any type of communication capabilities, such as RS232, RS485, Modbus TCP, MQTT, SLC, or any another wired or wireless protocol.

### Embodiments

The present invention can be embodied in a variety of ways. In one embodiment, the sensor is integrated into a sensor architecture that allows it to be connected to a controller (gateway, hub) to take decisions based on the generated data. In another embodiment, the sensor is able to operate on its own within one device and not requiring a controller.

In the preferred embodiment of the present invention, the multi-sensor is a compact, self-contained device that can be easily integrated into a variety of systems such as but not limited to stationary energy storage systems. The sensor includes a VOC sensor, an H2 sensor, a self-calibration mechanism, dry contact or relay outputs (optional), and communication capabilities.

The VOC sensor is a semiconductor sensor that is sensitive to a wide range of VOCs. The H2 sensor uses a newer hydrogen detection method, such as an optical sensor or a photo-ionization detector (PID). The self-calibration mechanism is a mechanism that uses a known gas concentration to calibrate the sensor. The dry contact or relay outputs are used to control external systems, such as vents or alarms. The communication capabilities are RS485, SLC, or another wired or wireless protocol.

The multi-sensor of the present invention is a cost-effective and reliable solution for detecting off-gas events from batteries. The sensor can be used to prevent fires and explosions in a variety of stationary energy storage systems.

### Benefits of the Invention

The present invention provides several benefits over prior art sensors, including:
Reduced cost: The multi-sensor of the present invention combines two sensors into a single device, which reduces the cost of purchase and installation.
Increased reliability: The mechanisms of the present invention eliminate the need for in-field calibration, which reduces the cost of ownership.
Improved safety: The multi-sensor of the present invention can detect all three types of off-gas events, which reduces the risk of fires and explosions.
Reduced complexity: The multi-sensor of the present invention can be operated with or without dry contact or relay outputs, which simplifies installation and maintenance.

The present invention is a novel and non-obvious solution to the problem of detecting off-gas events from batteries. The sensor is cost-effective, reliable, safe, and easy to use. The sensor has the potential to save lives and property by preventing fires and explosions in stationary energy storage systems.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

What has been described and illustrated herein is a preferred embodiment of the present invention along with some of its variations. The terms, descriptions and figures used herein are set forth by way of illustration only and are not meant as limitations. Those skilled in the art will recognize that many variations are possible within the spirit and scope of the present invention in which all terms are meant in their broadest, reasonable sense unless otherwise indicated. Any headings utilized within the description are for convenience only and have no legal or limiting effect.

Thus, it is appreciated that the optimum dimensional relationships for the parts of the present invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the present invention should be determined by the appended claims and their legal equivalents, rather than by the examples given.

The embodiments of the present invention in which an exclusive property or privilege is claimed are defined as follows:

1. A multi-sensor for detecting off-gas events from batteries, comprising:
a. a VOC sensor for detecting volatile organic compounds;
b. an H2 sensor for detecting hydrogen;
c. a self-calibration mechanism for calibrating the sensor without the need for human intervention;
d. dry contact or relay outputs (optional) for controlling external systems; and
e. communication capabilities for connecting to other systems.

2. The multi-sensor of claim 1, wherein the VOC sensor component is capable of detecting VOCs in the air.

3. The multi-sensor of claim 1, wherein the H2 sensor uses any type of hydrogen detection method.

4. The multi-sensor of claim 1, wherein the calibration free mechanism uses a gas component that is not vulnerable to drifting.

5. The multi-sensor of claim 1, wherein the self-calibration mechanism uses data from built-in or external sensors to calibrate itself.

6. The multi-sensor of claim 1, wherein optionally dry contact and/or relay outputs are used to systems such as but not limited to control vents or alarms.

7. The multi-sensor of claim 1, wherein the communication capabilities are RS232, RS485, Modbus TCP, MQTT, SLC, or any another wired or wireless protocol.

8. The multi-sensor of claim 1, wherein the sensor can optionally be integrated into a sensor architecture that allows it to be connected to a controller (gateway, hub) to take decisions based on the generated data.

9. The multi-sensor of claim 1, wherein the sensor is able to operate on its own within one device and not requiring a controller.

10. A method for processing H2 and VOC sensor data with possible other sensing data, comprising the steps of:
selecting one or more VOC sensors;
selecting one or more H2 sensors;
reading the metric data from the VOC and H2 sensors;
processing all data to generate an outcome; and
applying automatic corrective actions.

11. The method of claim 10, wherein the VOC sensors are a semiconductor sensor or an electrochemical sensor.

12. The method of claim 10, wherein the H2 sensors are hydrogen detection sensors are a metal oxide semiconductor (MOS) sensor, an electrochemical sensor, or a spectrometer.

13. The method of claim 10, further comprising the step of
receiving data from one or more other embedded or external sensing devices.

14. The method of claim 10, wherein automatic corrective actions include triggering a vent or stopping batteries.

15. The method of claim 10, further comprising the step of
sharing data with 3^{rd} party systems.

16. The method of claim 15, wherein the data is shared over industrial protocols, SLC or other communications systems.

17. The method of claim 10, further comprising the step of
executing a self-calibration protocol for calibrating the sensors without the need for human intervention.
